(19) United States
(12) Patent Application Publication
(10) Pub. No.: US 2010/0258502 A1
(43) Pub. Date: Oct. 14, 2010

(54) Advanced Oxidation Enhancements and High Temperature treatment of Contaminated Media

(76) Inventors: Brian E. Butters, London (CA); Anthony L. Powell, London (CA)

Correspondence Address: BAKER & MCKENZIE LLP;PATENT DEPARTMENT, 2001 ROSS AVENUE, SUITE 2300, DALLAS, TX, 75201, US

(21) Appl. No.: 12/701,439

(22) Filed: Feb. 5, 2010

Related U.S. Application Data
(60) Provisional application No. 61/150,661, filed on Feb. 6, 2009.

(51) Int. Cl. C02F 9/12; C02F 1/32; C02F 1/28; C02F 1/72; C02F 1/44; B01D 65/02

(52) U.S. Cl. 210/636; 210/205; 210/202; 210/195.2; 210/665

(57) ABSTRACT

Disclosed herein are decontamination techniques that use a chemical that acts as an oxygen source and adsorption/binding agent in a UV reactor to bring together organic contaminant molecules and TiO₂ molecules in the photoreactive slurry, or to alternatively bind directly to the contaminant molecules if no photocatalyst is employed. In one embodiment, one such system may comprise a contaminated fluid media source providing a contaminated fluid media, which could even be at near-boiling conditions. In addition, the decontamination system may include an adsorption accelerant comprising oxygen and soluble in the fluid media. The adsorption accelerant adsorbing to contaminant molecules in the contaminated fluid media, and should be stable if the fluid media is provided at near-boiling conditions. Also, such a decontamination system may include an irradiation source configured to irradiate the contaminated media containing the adsorption accelerant to eliminate the contaminant molecules from the fluid media.

### RELATED APPLICATIONS

The present application claims priority to provisonal Application Ser. No. 61/150,661, filed Feb. 6, 2009, the entire contents which are incorporated herein in its entirety, for all purposes.

### BACKGROUND

Steam assisted gravity distillation (SAGD) techniques are currently used in a number of subterranean applications, such as mining. Various industrial process streams, such as the recovery of steam condensate in SAGD operations, require decontamination treatment at very high water temperatures because of the desire to retain the energy for reuse in the production of steam in SAGD operations. For SAGD operations in oil mining, the steam condensate and bitumen are collected at near boiling levels. Processing the contaminated water stream at these high temperatures (i.e., without cooling the stream) are desired as it saves energy required to heat the water back up for steam generation as the SAGD operation continues.

One of the key issues with the treatment of SAGD steam condensate is that the water contains a portion of high molecular weight organics, which do not flash off in the boiler. These organics therefore stay in the boiler and cause organic fouling on the walls of the boiler (sometimes referred to as “coking”). These non-volatile organics (i.e., non-volatile TOC or NVTOC) are not sufficiently removed in typical SAGD water treatment technologies (i.e., de-oiling technologies, warm lime-softening, or evaporation technologies) due to entrainment of NVTOCs. Instead, what is required is an oxidation technology to break apart the high molecular weight compounds in the high-temperature contaminated fluid stream into smaller molecular weight, more volatile organic contaminants.

However, oxidizing the organics at high temperatures is typically problematic. Specifically, for this type of process an oxidant is required in the contaminated fluid stream. However, conventionally available advanced oxidation processes are rendered inefficient by the high-temperatures of the contaminated stream. Dissolved oxygen is normally used at ambient temperatures (<30° C.) as an oxidant; however, at the typical high-temperatures of >80° C. (if the contaminated fluid media is water) associated with processes such as SAGD, there is minimal dissolved oxygen in the water stream due to gas solubility at these high temperatures. Consequently, using dissolved oxygen would be a very inefficient process for high-temperature fluid streams. The same problem exists with employing ozone as an oxidant at these high temperatures.

Alternatively, the use of a chemical oxidant like hydrogen peroxide (H₂O₂) solves this solubility problem. However, at these high temperatures, peroxide is very unstable and will degrade rapidly. This creates a substantial safety hazard, and thus hydrogen peroxide is not an available option to treat contaminated media at high temperatures. Moreover, hydrogen peroxide also contains stabilizers which may be detrimental to boilers. Consequently, conventionally available processes cannot provide sufficient oxidation with typical UV irradiation, UV+hydrogen peroxide (H₂O₂) photolytic processes, UV+Ozone (O₃) photolytic processes, or even UV+Ozone & hydrogen peroxide photolytic decontamination processes, unless the fluid is sufficiently cooled. However, as mentioned above, significant energy is then waste re-heating the fluid back up for the SAGD process after decontamination with some of these conventionally available techniques.

### SUMMARY

Based on the above information, the problem of decontaminating a fluid media at elevated temperatures such as those associated with SAGD techniques is a two-part problem:
1. Providing an oxygen source required for oxidation at elevated temperatures such as those found in SAGD techniques; and
2. The oxygen additive must be safe to handle and process at such elevated temperatures.
   To overcome these issues, the decontamination technique disclosed herein has been developed. The disclosed technique includes salts to provide an oxygen source, and that provide the stability requirement for high temperature oxidation if the contaminated media is to be decontaminated at its near-boiling conditions.

Research and experience by the present inventors indicates that a limiting parameter in photocatalysis is the ability for the organic contaminants, the photoreactive catalyst (e.g., TiO₂ in a photocatalytic slurry), and the oxygen to come together at the same time the catalyst is irradiated with UV light. The collection of these four steps or components is generally required for a photocatalytic reaction to take place to destroy the contaminants and thereby decontaminate media in a reactor. However, the usual need for all components to come together simultaneously is also a rate-limiting parameter for photocatalysis. Various embodiments of such photocatalytic processes, as well as novel techniques and equipment, are disclosed in issued patents and currently pending patent applications co-owned with the present disclosure. For example, proprietary photocatalytic technology the present inventors has developed has increased photocatalytic performance by enhanced collision of TiO₂ and contaminants within a UV reactor using novel techniques for increasing turbulence in the reactor. See, e.g., U.S. Pat. No. 7,425,272, which is commonly assigned with the present disclosure and incorporated herein by reference in its entirety. Based on this enhancing of collision, higher rates of decontamination are achieved at higher mixing or turbulence.

Disclosed herein are decontamination techniques that build on this principle. Specifically disclosed is the use of a material or compound that acts as an oxygen source and adsorption/binding agent in a UV reactor to bring together organic contaminant molecules and TiO₂ molecules in the photoreactive slurry, or alternatively to bind directly to the contaminant molecules if no photoreactant or photocatalyst is employed. Thus, introducing such a specific material selected for these newly discovered properties reduces or eliminates the reliance on timely collision probability of the contaminant, TiO₂ and oxygen molecules by actively increasing the attraction between the organic contaminants and photoreactive TiO₂ molecules in photocatalytic applications. More generally, however, the selected materials disclosed herein provide an oxygen source for a photolytic reaction, without the need for oxygen-rich chemicals like hydrogen peroxide.

More specifically, the present inventors have discovered that oxygen-rich salts, such as compounds containing peroxymonosulfate (e.g., Oxone, Caroat, etc.), can provide such an active attraction between these specific molecules, or are simply actively attracted to contaminant molecules such as organic contaminants (e.g., VOCs), as well as providing an oxygen source for decontamination using a UV light source. Moreover, such oxygen-rich salts also provide an oxygen source useable in a decontamination process for fluid media at high temperatures, such as those associated with a SAGD process. In such applications, the selected adsorption accelerant must be stable (e.g., safe) at the near-boiling temperatures and pressures for the contaminated fluid media being decontaminated. If the contaminated media is water, the adsorption accelerant must be stable at temperatures approaching up to the boiling point of water, e.g., 80-99° C. or greater if the water is pressurized. Accordingly, the disclosed principles herein not only provide for a beneficial technique using an adsorption accelerant to bind directly to contaminants or to accelerate/facilitate binding between contaminants and a photoreactant, and provide oxygen in a UV light-based decontamination process, but also provide a chemical-free technique usable in high-temperature decontamination applications.

Therefore, in view of the above, in one aspect, decontamination systems for decontaminating fluid media having contaminant molecules, even high-temperature fluid media, are disclosed herein. In one embodiment, one such system may comprise a contaminated fluid media source providing a contaminated fluid media. In addition, the decontamination system may include an adsorption accelerant comprising oxygen and soluble in the fluid media, and it is the adsorption accelerant that binds to contaminant molecules in the contaminated fluid media. In embodiments where the contaminated fluid media source is provided at near-boiling levels, the adsorption accelerant should be stable at the near-boiling levels of the fluid media. Also, such a decontamination system would include an irradiation source configured to irradiate the contaminated media containing the adsorption accelerant to eliminate the contaminant molecules from the fluid media.

In a more specific embodiment, a decontamination system according to the disclosed principles may comprise a contaminated fluid media source providing a contaminated fluid media, and a photocatalytic system including a photoreactant or photocatalyst. In addition, such an exemplary system may comprise a salt-based adsorption accelerant comprising oxygen and soluble in the fluid media. Again, in embodiments where the contaminated fluid media source is provided at near-boiling levels, the adsorption accelerant should be stable at the near-boiling temperatures of the fluid media. Since a photocatalyst, such as TiO₂ is present, the adsorption accelerant binds contaminant molecules and photocatalyst molecules together. Furthermore, such a system would include a UV light source associated with the photocatalytic system and configured to irradiate the contaminated media containing the bound adsorption accelerant, contaminant molecules and photocatalyst molecules to eliminate the contaminant molecules from the fluid media.

In another aspect, methods of decontaminating fluid media are disclosed. In one embodiment, such a method may comprise providing a contaminated fluid media, such as contaminated water, and may even provide the fluid media at high, near-boiling temperatures and pressures. In addition, such a method would include adding an adsorption accelerant comprising oxygen and soluble to the fluid media, where the adsorption accelerant binds to contaminant molecules in the contaminated fluid media. Further, such an exemplary method would include irradiating the contaminated media containing the adsorption accelerant to eliminate the contaminant molecules from the fluid media. If a photocatalytic reaction is desired, a photoreactant/photocatalyst may also be introduced in the contaminated media, and the adsorption accelerant would bind the photocatalyst to the contaminant molecules. If the contaminated media is provided at near-boiling temperature, the adsorption accelerant would be stable at the near-boiling temperatures of the fluid media.

### BRIEF DESCRIPTION OF THE DRAWINGS

Reference is now made to the following detailed description of the preferred embodiments, taken in conjunction with the accompanying drawings. It is emphasized that various features may not be drawn to scale. In fact, the dimensions of various features may be arbitrarily increased or reduced for clarity of discussion. In addition, it is emphasized that some components may not be illustrated for clarity of discussion. Reference is now made to the following descriptions taken in conjunction with the accompanying drawings, in which:

FIG. 1 illustrates one embodiment of a decontamination system providing the disclosed principles integrated into a water decontamination process for contaminated fluids; and

FIG. 2 illustrates another embodiment of a decontamination system constructed and implemented in accordance with the disclosed principles.

### DETAILED DESCRIPTION

The novel solution disclosed herein involves adding a soluble salt to an advanced oxidation process, and specifically a salt that incorporates significant amounts of oxygen in its molecular structure. This specially selected salt, which is soluble and stable at high temperatures, is able to provide the required oxygen to the advanced oxidation process for decontamination of media without the dangers and added expense of using oxygen containing chemicals such as hydrogen peroxide, and without the added expense of additional systems such as ozone-generating systems. Additionally, the disclosed technique is also applicable at the extreme temperatures and conditions discussed in above. Examples of such a salt include Oxone, Caroat, persulphate, peroxyphosphate and other materials or compounds containing peroxymonosulfate. These types of salts are safe to handle at high temperatures (i.e., >80° C. for water) and allow advanced oxidation process water purification at such elevated temperatures with no practical up-limit. Even more generally, such salt-based materials act as adsorption accelerants and thus accelerate binding of the material's molecules to contaminant molecules (e.g., volatile organic contaminants (VOCs)), or facilitate the binding of contaminant molecules and molecules of a photocatalyst.

Furthermore, the addition of such oxygen-rich salts to the high-temperature contaminated media will not add dissolved oxygen to the stream. If dissolved oxygen is added to a stream, such as in conventional oxidation processes mentioned above, the dissolved oxygen must be later removed from the stream for Boiler Feed Water (BFW) (e.g., in SAGD operations). Besides the inefficiency of adding dissolved oxygen at high temperatures required for oxidation, subsequent removal of residual dissolved oxygen required for BFW is a difficult and expensive process. Thus, in accordance with the disclosed principles, processing the water at even high a temperatures still minimizes dissolved oxygen contamination due to the low solubility of the disclosed materials, which will reduce the amount and cost of deoxygenating the water before it enters the boilers.

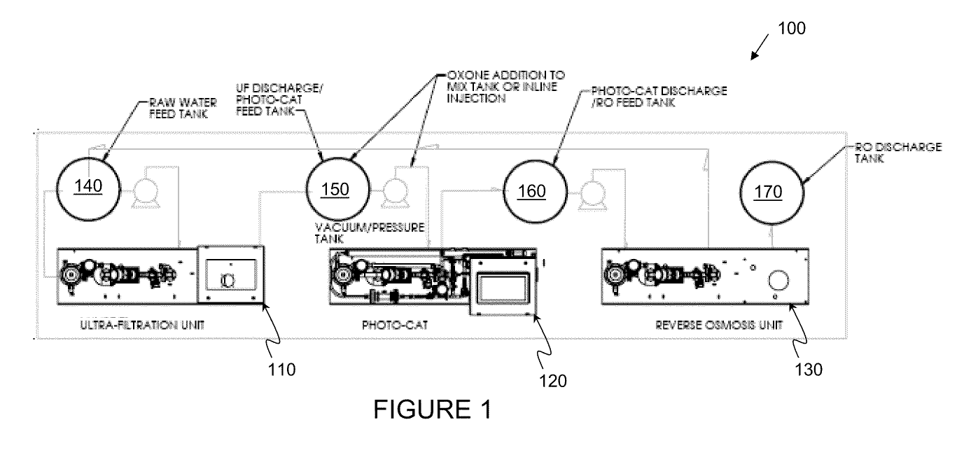

FIG. 1 illustrates how the disclosed principles may be integrated into a water decontamination process for contaminated fluids. In addition, the disclosed principles may be employed for high-temperature contaminated fluids, such as the high temperatures associated and employed for SAGD processes. As shown, an exemplary decontamination system 100 may include three phases. Phase 1 of a high temperature decontamination process conducted in accordance with the disclosed principles may include the ultra-filtration of the incoming contaminated feed stream (e.g., water) with an ultra-filtration unit 110. The filtered stream may then move to Phase 2, which not only includes the photocatalytic reactor 120 (and accompanying photocatalyst slurry), but also includes the peroxymonosulphate (or similarly behaving salt- or peroxymonosulfate-containing material) addition technique disclosed herein. Phase 3 of the disclosed exemplary embodiment may then include a Reverse Osmosis system 130 for the removal or unused portions of the salt-based materials employed in the disclosed technique and other entrained Total Dissolved Solids).

Looking at FIG. 1 in additional detail, the ultra-filtration unit 110 may be provided the contaminated fluid to be decontaminated from a raw feed water tank 140. In accordance with the disclosed principles, the contaminated water or other media may be at near boiling levels (depending upon temperature and pressure), which are the conditions typically associated with SAGD process, as discussed above. Moreover, instead of a feed tank 140, the contaminated media may simply be directly flowed into the ultra-filtration system 110. Even further, an ultra-filtration system 110 is may not even be required for conducting a high-temperature decontamination process in accordance with the disclosed principles.

In order to integrate the ultra-filtration phase to a photocatalytic phase, a level-controlled vacuum tank 150 (e.g., the “accumulator” in the “Photo-Cat” equipment developed by the present inventors). This may also be a process or other equipment used to remove dissolved oxygen for BFW. For example, a small vacuum in a break tank may be pulled, which will help remove gas bubbles and dissolved oxygen in the media. It will also reduce power (e.g., horsepower) requirements in the feed pump to the Phase 1 ceramic filtration membranes. Furthermore, it can also act as a dampener for shockwaves sent to the photocatalytic equipment used to remove build up in the photocatalytic equipment.

In addition, the Phase 2 equipment pump may be used to pull (i.e., create a vacuum), which will reduce pumping requirements for the Phase 1 equipment. Alternatively, or additionally, if steam is used, which when it condenses in a tank it will create a vacuum, this may be more efficient than a pump or a pump alone. When these principles are implemented, a decontamination process as disclosed herein (even at high-temperatures) should allow the Phase 1 ultra-filtration equipment 110 to run for extended periods of time (i.e., months) before any chemical cleaning is required. Also, ultra-filtration in train eliminates the potential for oil upset from dissolved air flotation (DAF) or other pretreatment for gross oil removal is such applications.

During the Phase 2 photocatalytic decontamination process, discharged media from the ultra-filtration unit 110 maybe provided in the feed tank 150. Alternatively, the discharged, filtered media may be directly fed into the photocatalytic equipment 120. In either embodiment, in accordance with the disclosed principles, the addition of peroxymonosulphate or other similarly acting salt-based material is provided to the contaminated fluid media before the photocatalytic process. For example, if a feed tank 150 is employed in the system 100, the peroxymonosulphate or other oxygen-rich, soluble, high-temperature resistant additive may be added to the media while in the feed tank 150. The time the media (containing the contaminants) and additive spend in the tank 150 together can help promote the bonding or binding of the contaminant molecules and TiO₂ molecules found in the photocatalytic slurry.

Specifically, the peroxymonosulphate or similar salt-based additive acts as an oxygen source and adsorption/binding agent in a photocatalytic reactor to bring together organic contaminant molecules and TiO₂ (or other photocatalyst) molecules in the photoreactive slurry. Thus, introducing such a specific salt-based compound selected for these newly discovered properties reduces or eliminates the reliance on timely collision probability of the contaminant, TiO₂ and oxygen molecules by actively increasing the attraction between the organic contaminants and photoreactive TiO₂ molecules, and simultaneously provides an oxygen source for the photolytic reaction. Moreover, in addition to simply spending storage time in the feed tank 150 together, the tank 150 may also be equipped or configured to work with a turbulence or agitation system or process. The addition of such agitation, while not required to practice the disclosed principles, may further aid in the peroxymonosulphate or other additive promoting the binding of the contaminant molecules and the TiO₂ (or other photocatalyst) molecules.

In addition, the disclosed principles provide for the unused salt (e.g., peroxymonosulphate) and other dissolved solids to be removed by blow down or a reverse osmosis (RO) process in Phase 3. Specifically, after a photocatalytic decontamination process, the discharge from the photocatalytic unit 120 may be discharged to a reverse osmosis feed tank 160. Alternatively, the discharge from the photocatalytic system 120 may be directly fed into the RO unit 130.

A reverse osmosis process may be included since the disclosed technique eliminates the large particles (i.e., large molecular weight) that typically clog RO components. The sterile, low molecular weight feed to an RO stage prevents or significantly reduces fouling mechanisms therein, and the chemical cleaning requirements and failure mechanisms when large molecular weight particles are passed to an RO system. The incorporation of a RO process along with a system or process in accordance with the disclosed principles eliminates Total Dissolved Solids (TDS), and produces a pristine BFW, such that any type of boiler can be used (i.e., more efficient drum boilers) for SAGD or other applications. This can create huge savings in energy by reducing blow down fuel consumption. Also, the disclosed approach still allows for including a BFW treatment process if desired in order to consume the dissolved oxygen that may be present in the stream. After the reverse osmosis process has been completed, the decontaminated media may be output to an RO discharge tank 170, or may be discharged through another output of the system 100.

Looking specifically at the exemplary use of peroxymonosulphate in the disclosed process, it is known that peroxymonosulphate may be employed as an irreversible electron acceptor in a decontamination process. So too can hydrogen peroxide be used as an irreversible electron acceptor. However, the present inventors have discovered that peroxymonosulphate provides an adsorption phenomena when employed with TiO₂ or other similar photocatalyst for photocatalysis for decontaminating contaminants in water. This is demonstrated in that in tests performed, equal Molar ratios of peroxymonosulphate to H₂O₂ have an order of magnitude higher performance in photocatalytic processes. TABLE 1 below sets forth the data for such an exemplary test that was performed using potassium peroxymonosulfate (i.e., ADX) as the adsorption accelerant, and which shows the results of 1,4-dioxane destruction with a photocatalytic process in the decontamination system. The power for both tests were identical, with the primary difference between tests being that hydrogen peroxide was added in the first test, and potassium peroxymonosulfate was used in the other.

TABLE 1

| Flow | Peroxide | ADX | Influent | Effluent | 1st Order |
|---|---|---|---|---|---|
| (Lpm) | (ppm) | (ppm) | (ppb) | (ppb) | Rate (k) |
| 8 | 600 | 0 | 1830 | 443 | 1.1 |
| 10 | 0 | 172 | 2240 | 16 | 5.0 |

The data in TABLE 1 demonstrates the increased efficiency of a peroxymonosulfate-containing material. Specifically, the adsorption accelerant increased the rate constant (a means for comparing the tests to one another) almost 5 fold, and at a lower dosage than the hydrogen peroxide.

Thus, what has been discovered by the present inventors is that peroxymonosulphate and other salt-based materials offers more than just the function of an electron acceptor. It has a surfactant type property (e.g., like soap that brings the water and dirt together). For peroxymonosulphate, the peroxymonosulphate adsorbs to the TiO₂, and encourages adsorption of the organic contaminant molecules to the TiO₂ thereafter. More specifically, the peroxymonosulphate adsorbs to the TiO₂ molecules found in a photocatalytic slurry. This adsorption is independent of any high-turbulence that may be introduced in the decontamination system to promote contact of the two different molecules. Instead, the peroxymonosulphate is actively attracted to the TiO₂, and adsorbs to it without any additional promotion of their bonding. In addition, the peroxymonosulphate, once adsorbed to the TiO₂, also actively attracts organic contaminants to the TiO₂, and again this occurs without any reliance of timely collision probability like increasing turbulence in the reactor. Of course, in both cases, increasing turbulence may further the efficiency of the adsorptions in less time.

Also, a pressure or vacuum vessel may be included in a decontamination system constructed according to the disclosed principles to provide an area with the adsorption may occur. Alternatively, such a vessel may be placed between incoming contaminated media stream and the photocatalytic slurry, or in another advantageous location, of an existing system, and thus allow for integrating the disclosed principles into the existing system.

Once the contaminants are adsorbed to the peroxymonosulphate-laden TiO₂, UV irradiation is employed to cause a photocatalytic reaction with the TiO₂ and contaminants. At this point, the four parts for photocatalytic decontamination are present, namely, the contaminant, the photocatalyst (TiO₂), the oxygen source, and the UV light. The use of an oxygen-rich salt like peroxymonosulphate promotes the attraction/adhesion of the three different molecules, and thereby efficiently creates the combination for the UV light irradiation. In short, the disclosed principles make the photocatalytic reaction used to decontaminate the media happen easier and quicker, because of the adsorption property to both the TiO₂ and organic contaminants provided by the peroxymonosulphate. Any salt-based material incorporating peroxymonosulfate can be utilized. During this photocatalytic reaction, the contaminants are destroyed and the contaminated media stream purified. And all of these same principles may be applied to applications using a high-temperature contaminated media. In recent testing by the present inventors, TOCs in a SAGD water stream contaminated with bitumen were reduced from 650 ppm down to 300 ppm with just 645 mg/L of peroxymonosulphate. The ‘active oxygen’ part of peroxymonosulphate is only 5.2% (33.5 ppm) of the total peroxymonosulphate molecule. This shows that the ‘non-active oxygen’ components of peroxymonosulphate are being utilized in the photocatalytic process.

Furthermore, the adsorption of the peroxymonosulphate to the TiO₂ is maintained until it is consumed. Therefore, because the adsorption of the peroxymonosulphate to the TiO₂ is maintained, the disclosed principles also provide for the novel technique and process disclosed herein to be closed-loop. Specifically, the disclosed technique provides for the recycle and reuse of the unused (i.e., unreacted) photocatalytic slurry with the adsorbed peroxymonosulphate until it is consumed. Therefore, the TiO₂ photocatalyst is not immobilized in the reactor, and is instead recycled from the irradiation area of the reactor for use with incoming contaminated media. This results in a highly efficient closed-loop decontamination reactor, where a small amount of peroxymonosulphate or similar component added to a photocatalytic slurry, such as TiO₂, creates a powerful decontamination slurry orders of magnitude more efficient than merely employing an oxidant in a system, and is also capable of use in high-temperature decontamination applications.

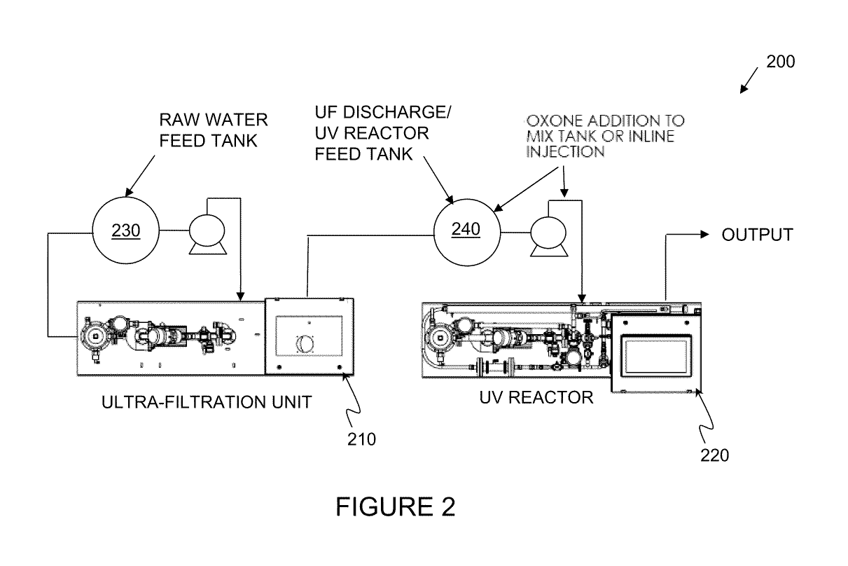

Even when not employed in high-temperature applications, however, the high efficiency created illustrates another advantage of peroxymonosulphate or similarly behaving material—that it may be used in decontaminating potable or drinking water, with a photocatalytic process or even with the use of UV irradiation alone without a photocatalyst. This is because the use of peroxymonosulphate results in benign byproducts that do not require post-treatment after decontamination of the potable stream. FIG. 2 illustrates a high-level block diagram of another embodiment of a system 200 constructed in accordance with the disclosed principles. This embodiment, however, differs from the system 100 in FIG. 1 in that it does not include a photocatalytic reactor. Instead, in such an embodiment, the disclosed principles are simplified into a technique that combines the use of a oxygen-rich salt-based material, such as peroxymonosulphate, with a UV irradiation source.

Looking specifically at FIG. 2, the high-temperature decontamination system 200 includes an ultra-filtration unit 210 and an ultraviolet (UV) light reactor unit 220. As before, the ultra-filtration unit 210 is optional in the system 200, but may be employed to initially filter out larger particles from the high-temperature contaminated fluid media. Also as before, the ultra-filtration unit 210 may be provided the contaminated fluid to be decontaminated from a raw feed water tank 230, and may again be provided at the typical high temperature, e.g., about 80-99° C., or more typically 90-95° C., associated with SAGD process. If the water is under pressure, which is often the case, then temperatures of the contaminated fluid media may even reach 120° C. or higher. Of course, instead of a feed tank 230, the contaminated media may simply be directly flowed into the ultra-filtration system 210.

To integrate the ultra-filtration phase to a UV reactor, another feed tank 240 may be employed. Again, this tank may be a level-controlled vacuum tank, or may be a process or other equipment used to remove dissolved oxygen for BFW. In accordance with the disclosed principles, the addition of peroxymonosulphate or other similarly behaving salt-based material is provided to the contaminated fluid media before the UV irradiation process. For example, if a feed tank 240 is employed in the system 200, the peroxymonosulphate or other salt-based oxygen-rich soluble high-temperature resistant additive may be added to the media while in the feed tank 240. The time the media (containing the contaminants) and additive spend in the tank 150 together can help promote the bonding or binding of the contaminant molecules and peroxymonosulphate or other adsorption accelerant.

Specifically, the peroxymonosulphate or similar additive acts as an oxygen source and binds in the UV reactor 220 to the organic contaminant molecules. Due do it's high oxygen content, the peroxymonosulphate or similar additive acts as the oxygen source for the photolytic reaction brought about by the UV irradiation. As before, in addition to simply spending storage time in the feed tank 240 together, the tank 240 may also be equipped or configured to work with a turbulence or agitation system or process. The addition of such agitation, while not required to practice the disclosed principles, may further aid in the peroxymonosulphate or other additive binding to the contaminant molecules. In addition, although not illustrated, this embodiment of a system 200 constructed according to the disclosed principles may also provide for the unused salt-based material, if any, to be removed by blow down or a reverse osmosis (RO) process in a third phase to reduce or eliminate dissolved solids.

Regardless of the embodiment, when the disclosed principles are also employed in high-temperature applications, amalgam lamps may also be employed for their ability to perform efficiently at high fluid temperatures. In addition, ceramic membranes for oil removal may also be employed based on another recognition that there is no biological fouling of the ceramic membranes and their ability to operate at elevated temperatures and pressures. Moreover, in such applications, the clay or other suspended solids in the contaminated stream provides scouring or honing for the ceramic membranes, which typically solves the fouling mechanism problem. Thus, the presence of such suspended solids in such a closed-loop system is advantageous with techniques implemented in accordance with the disclosed principles, which is contrary to traditional membrane-based filtration systems. In addition, a back-pulse or ultrasonic treatment (i.e., an instantaneous shock or hammer) of the ceramic membranes in the ultra-filtration system may also be employed to prevent any build-up on the membranes, as opposed to removing built-up layers after forming on the membranes. Such technique is disclosed in our U.S. patent application Ser. No. 11/681,555, which is also incorporated herein by reference in its entirety.

While various embodiments in accordance with the disclosed principles have been described above, it should be understood that they have been presented by way of example only, and are not limiting. Thus, the breadth and scope of the invention(s) should not be limited by any of the above-described exemplary embodiments, but should be defined only in accordance with the claims and their equivalents issuing from this disclosure. Furthermore, the above advantages and features are provided in described embodiments, but shall not limit the application of such issued claims to processes and structures accomplishing any or all of the above advantages.

Additionally, the section headings herein are provided for consistency with the suggestions under 37 C.F.R. 1.77 or otherwise to provide organizational cues. These headings shall not limit or characterize the invention(s) set out in any claims that may issue from this disclosure. Specifically, a description of a technology in the “Background” is not to be construed as an admission that technology is prior art to any invention(s) in this disclosure. Neither is the “Summary” to be considered as a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings herein.

1. A decontamination system for decontaminating fluid media, the system comprising:
a contaminated fluid media source providing a fluid media contaminated with contaminant molecules;
an adsorption accelerant comprising oxygen and soluble in the fluid media, the adsorption accelerant adsorbing to contaminant molecules in the contaminated fluid media; and
an irradiation source configured to irradiate the contaminated media containing the adsorption accelerant to eliminate the contaminant molecules from the fluid media.

2. A decontamination system according to claim 1, wherein the adsorption accelerant is a salt-based material.

3. A decontamination system according to claim 2, wherein the salt-based adsorption accelerant comprises peroxymonosulfate.

4. A decontamination system according to claim 1, wherein the fluid media is a high-temperature fluid media provided at near-boiling temperature, and wherein the adsorption accelerant is stable at the near-boiling conditions of the fluid media.

5. A decontamination system according to claim 1, wherein the irradiation source is an ultraviolet light source.

6. A decontamination system according to claim 1, further comprising an ultra-filtration system having ceramic filtering membranes configured to filter the contaminated media prior to the irradiation source.

7. A decontamination system according to claim 6, wherein the system is a closed-loop system and the contaminated fluid media further comprises suspended solids, the suspended solids providing scouring or honing for ceramic membranes in the ultra-filtration system.

8. A decontamination system according to claim 6, wherein the ultra-filtration system further comprises a shockwave generating unit configured to deliver an instantaneous shock to the ceramic membranes in the ultra-filtration system to prevent contaminant build-up on the ceramic membranes.

9. A decontamination system according to claim 1, further comprising a reverse osmosis system configured to remove Total Dissolved Solids (TDS) from the irradiated fluid media.

10. A decontamination system according to claim 1, further comprising a photocatalytic system incorporating the irradiation source and including a photocatalyst, wherein the adsorption accelerant adsorbs to the contaminant molecules and molecules of the photocatalyst to facilitate binding of the contaminant molecules and photocatalyst molecules together prior to irradiation.

11. A decontamination system according to claim 10, wherein the photocatalyst is TiO₂.

12. A decontamination system for decontaminating fluid media, the system comprising:
a contaminated fluid media source providing a fluid media contaminated with contaminant molecules;
a photocatalytic system including a photocatalyst;
a salt-based adsorption accelerant comprising oxygen and soluble in the fluid media, wherein the adsorption accelerant adsorbs to the contaminant molecules and molecules of the photocatalyst to facilitate binding of the contaminant molecules and photocatalyst molecules together;
a UV light source associated with the photocatalytic system and configured to irradiate the contaminated media containing the bound adsorption accelerant, contaminant molecules and photocatalyst molecules to eliminate the contaminant molecules from the fluid media.

13. A decontamination system according to claim 12, wherein the salt-based adsorption accelerant comprising peroxymonosulfate.

14. A decontamination system according to claim 12, wherein the fluid media is a high-temperature fluid media provided at near-boiling temperature, and wherein the adsorption accelerant is stable at the near-boiling temperature of the fluid media.

15. A decontamination system according to claim 12, further comprising an ultra-filtration system having ceramic filtering membranes configured to filter the contaminated media prior to the irradiating.

16. A decontamination system according to claim 15, wherein the system is a closed-loop system and the contaminated fluid media further comprises suspended solids, the suspended solids providing scouring or honing for ceramic membranes in the ultra-filtration system.

17. A decontamination system according to claim 15, wherein the ultra-filtration system further comprises a shockwave generating unit configured to deliver an instantaneous shock to the ceramic membranes in the ultra-filtration system to prevent contaminant build-up on the ceramic membranes.

18. A decontamination system according to claim 12, further comprising a reverse osmosis system configured to remove Total Dissolved Solids (TDS) from the irradiated fluid media.

19. A decontamination system according to claim 12, wherein the photocatalyst is TiO₂.

20. A method of decontaminating fluid media, the method comprising:
providing a fluid media contaminated with contaminant molecules;
adding an adsorption accelerant comprising oxygen and soluble to the fluid media, the adsorption accelerant adsorbing to contaminant molecules in the contaminated fluid media; and
irradiating the contaminated media containing the adsorption accelerant to eliminate the contaminant molecules from the fluid media.

21. A method according to claim 20, wherein the adsorption accelerant is a salt-based material.

22. A method according to claim 21, wherein the salt-based adsorption accelerant comprises peroxymonosulfate.

23. A method according to claim 20, wherein the fluid media is provided at a near-boiling temperature, and wherein the adsorption accelerant is stable at the near-boiling conditions of the fluid media.

24. A method according to claim 20, wherein irradiating comprises irradiating with ultraviolet light.

25. A method according to claim 20, further comprising filtering the contaminated media prior to the irradiating.

26. A method according to claim 25, wherein the filtering comprises filtering using an ultra-filtration system having ceramic filtering membranes, and the contaminated fluid media includes suspended solids, the method further comprising scouring or honing the ceramic membranes by flowing the suspended solids against the ceramic membranes in the ultra-filtration system.

27. A method according to claim 25, wherein the filtering further comprises delivering an instantaneous shockwave to the ceramic membranes in the ultra-filtration system to prevent contaminant build-up on the ceramic membranes.

28. A method according to claim 20, removing Total Dissolved Solids (TDS) from the irradiated fluid media.

29. A method according to claim 20, further comprising adding a photocatalyst to the contaminated media prior to the irradiating, wherein the adsorption accelerant adsorbs to the contaminant molecules and molecules of the photocatalyst to facilitate binding of the contaminant molecules and photocatalyst molecules together prior to the irradiating.

30. A method according to claim 29, wherein the photocatalyst is TiO₂.
